(19) United States
(12) Patent Application Publication
(10) Pub. No.: US 2021/0259529 A1
(43) Pub. Date: Aug. 26, 2021

(54) GUIDE TUBE, TREATMENT SYSTEM AND GUIDE-TUBE ATTACHMENT METHOD

(71) Applicant: OLYMPUS CORPORATION, Tokyo (JP)

(72) Inventors: Ryoji HYODO, Tokyo (JP); Koji NISHIZAWA, Tokyo (JP)

(73) Assignee: OLYMPUS CORPORATION, Tokyo (JP)

(21) Appl. No.: 17/318,385

(22) Filed: May 12, 2021

Related U.S. Application Data
(63) Continuation of application No. PCT/JP2018/043101, filed on Nov. 22, 2018.

(51) Int. Cl. A61B 1/00; A61B 1/018

(52) U.S. Cl. CPC A61B 1/00135; A61B 1/0014; A61B 1/00154; A61B 1/018

(57) ABSTRACT

Provided is a guide tube including an elongated sheath into which an elongated treatment tool is inserted so as to be movable in a longitudinal direction and that is arranged so as to be parallel to an endoscope, wherein: the sheath has a first end having a projection port from which the treatment tool projects and a second end having an insertion port into which the treatment tool is inserted and is divided, in the longitudinal direction, into a first region including the first end and having flexibility and a second region including the second end; the second region has a high-rigidity portion having higher rigidity than a rigidity of the first region; and a lengths of the first region is not greater than a length of the second region.

### CROSS-REFERENCE TO RELATED APPLICATIONS

This is a continuation of International Application PCT/JP2018/043101 which is hereby incorporated by reference herein in its entirety.

### TECHNICAL FIELD

The present invention relates to a guide tube, a treatment system and a guide-tube attachment method, and particularly relates to a guide tube for a treatment tool used in combination with an endoscope.

### BACKGROUND ART

In the related art, there is a known treatment-tool tube that is attached to the outside of an endoscope (for example, see Patent Literature 1).

### CITATION LIST

### Patent Literature

{PTL 1} Japanese Unexamined Patent Application, Publication No. 2013-188638

### SUMMARY OF INVENTION

An aspect of the present invention is a guide tube including an elongated sheath into which an elongated treatment tool is inserted so as to be movable in a longitudinal direction and that is arranged so as to be parallel to an endoscope, wherein: the sheath has a first end having a projection port from which the treatment tool projects and a second end having an insertion port into which the treatment tool is inserted and is divided, in the longitudinal direction, into a first region including the first end and having flexibility and a second region including the second end; the second region has a high-rigidity portion having higher rigidity than a rigidity of the first region; and a lengths of the first region is not greater than a length of the second region.

Another aspect of the present invention is a guide tube including an elongated sheath into which an elongated treatment tool is inserted in a longitudinal direction and that is arranged so as to be parallel to an endoscope, wherein: the sheath has a first end from which the treatment tool projects and a second end into which the treatment tool is inserted and is divided, in the longitudinal direction, into a first region including the first end and having flexibility and a second region including the second end; the second region has a high-rigidity portion having higher rigidity than a rigidity of the first region; and the high-rigidity portion includes a length adjusting mechanism.

Another aspect of the present invention is a treatment method using a guide tube including an elongated sheath into which an elongated treatment tool is inserted in a longitudinal direction and that is arranged so as to be parallel to an endoscope, the sheath being divided, in the longitudinal direction, into a first region including a first end from which the treatment tool projects and having flexibility, and a second region including a second end into which the treatment tool is inserted and having a high-rigidity portion having higher rigidity than a rigidity of the first region, the method including: inserting only the first region into a body cavity; and rotationally operating the treatment tool after the inserting.

### BRIEF DESCRIPTION OF DRAWINGS

FIG. 1 is a partial configuration diagram of a treatment system according to an embodiment of the present invention.

FIG. 2A is a schematic side view of a guide tube in FIG. 1.

FIG. 2B is a perspective view of a configuration example of a fixing portion of the guide tube in FIG. 1.

FIG. 3 is a diagram showing a usage example of the treatment system in FIG. 1.

FIG. 4A is a partial longitudinal sectional view showing a configuration example of a sheath.

FIG. 4B is a partial side view showing another configuration example of the sheath.

FIG. 4C is a partial longitudinal sectional view showing another configuration example of the sheath.

FIG. 4D is a partial side view showing another configuration example of the sheath.

FIG. 5A is a longitudinal sectional view showing a configuration example of a length adjusting mechanism in an extended state.

FIG. 5B is a longitudinal sectional view showing the length adjusting mechanism in FIG. 5A in a contracted state.

FIG. 6A is a longitudinal sectional view showing another configuration example of the length adjusting mechanism in an extended state.

FIG. 6B is a longitudinal sectional view showing the length adjusting mechanism in FIG. 6A in a contracted state.

FIG. 7 is a longitudinal sectional view showing a configuration of a modification of the sheath.

FIG. 8 is a partial configuration diagram of a treatment system showing another modification of the sheath.

FIG. 9 is a partial configuration diagram of a treatment system showing another modification of the sheath.

### DESCRIPTION OF EMBODIMENT

A guide tube 1 and a treatment system 100 according to an embodiment of the present invention will be described below with reference to the drawings.

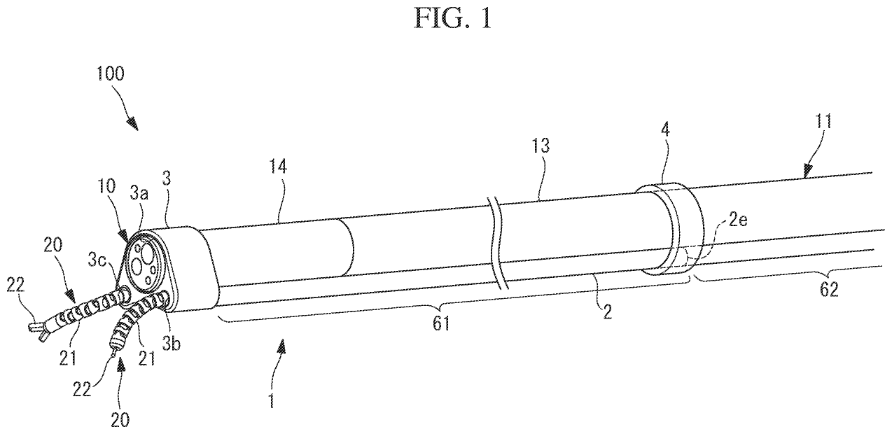

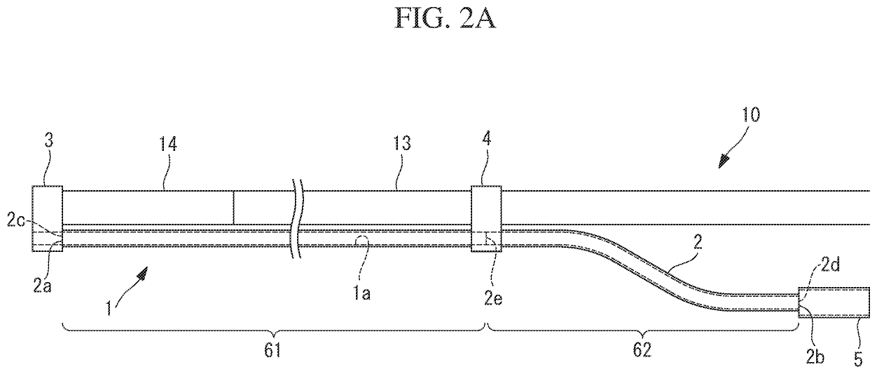

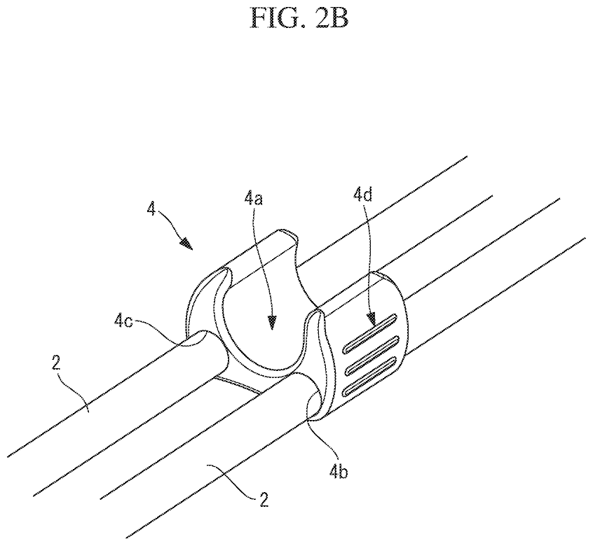

As shown in FIGS. 1 to 3, the treatment system 100 according to this embodiment includes an endoscope 10, a treatment tool 20, and the guide tube 1 that guides the treatment tool 20. In an example in FIGS. 1 to 2B, two treatment tools 20 are guided by two sheaths 2 of the guide tube 1. In the following, the guide tube 1 including two sheaths 2 and the treatment system 100 including two treatment tools 20 will be described. However, the numbers of the sheath 2 and the treatment tool 20 may both be only one or three or more.

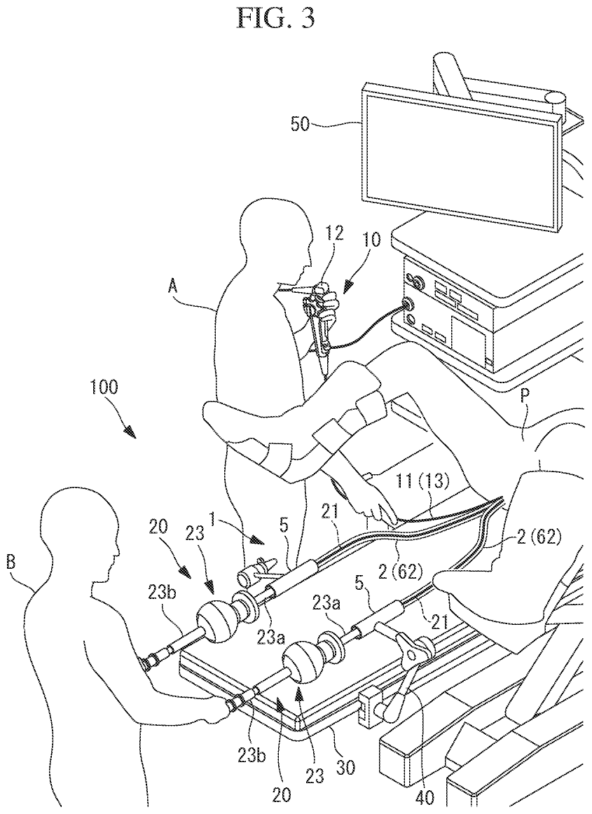

FIG. 3 shows a usage state of the treatment system 100. A surgical arm 40 for supporting a holder 5 (described later) of the guide tube 1 is fixed to a surgical bed 30 on which a patient P lies. As a usage example of the treatment system 100, FIG. 3 shows an example in which the endoscope 10 and the guide tube 1 are inserted into the large intestine from the anus of the patient P. The treatment tools 20 are inserted through the guide tube 1 to an affected site in the body cavity of the patient P. Reference sign 50 indicates a display for displaying an endoscope image observed with the endoscope 10.

The endoscope 10 is a flexible endoscope that is inserted into a body cavity such as the large intestine. The endoscope 10 includes an elongated insertion portion 11 and an operating portion 12 that is connected to the proximal end of the insertion portion 11. The insertion portion 11 has an elongated flexible portion 13 having flexibility and a bending portion 14 that is connected to the distal end of the flexible portion 13. A scopist A can control a bending operation of the bending portion 14 by operating the operating portion 12.

The treatment tools 20 each include: an elongated insertion portion 21 having flexibility; an end effector 22 that is connected to the distal end of the insertion portion 21; and a manipulating portion 23 that is connected to the proximal end of the insertion portion 21. The end effector 22 is a portion that applies treatment to biological tissue and is, for example, an electrode or forceps.

As shown in FIG. 3, the manipulating portion 23 includes: a shaft 23a that is connected to the proximal end of the insertion portion 21 and that has a larger diameter than the insertion portion 21; and a manipulating member 23b that is connected to the proximal end of the shaft 23a and that can be swiveled with respect to the shaft 23a. A distal end portion of the insertion portion 21 is configured to bend in accordance with an inclination of the manipulating member 23b relative to the shaft 23a. An operator B can advance and retract the insertion portion 21 and the end effector 22 by advancing and retracting the manipulating portion 23 in a longitudinal direction. In addition, the operator B can rotate the insertion portion 21 and the end effector 22 by rotating the manipulating portion 23 about a longitudinal axis of the insertion portion 21.

The guide tube 1 includes: the two elongated, tubular sheaths 2 that are parallel to each other; a cap 3 that is fixed to distal ends 2a of the two sheaths 2; a fixing portion 4 that is attached to intermediate positions of the sheaths 2 in the longitudinal direction and that is for fixing the two sheaths 2 to the insertion portion 11; and cylindrical rigid holders 5 that are connected to proximal ends 2b of the respective sheaths 2. In addition, the guide tube 1 has two channels 1a that penetrate from a distal end surface of the cap 3 to proximal end surfaces of the holders 5 through the interiors of the respective sheaths 2 and into which the insertion portions 21 and the end effectors 22 of the treatment tools 20 are inserted. The inner diameter of each of the channels 1a is larger than the outer diameters of the insertion portion 21 and the end effector 22, and the insertion portion 21 can advance and retract in the longitudinal direction and can also rotate about the longitudinal axis in the channel 1a.

The cap 3 has three holes 3a, 3b, 3c that penetrate in a direction along the longitudinal axis of the sheath 2. One hole 3a is for the endoscope 10 and can be fitted with a distal end portion of the endoscope 10. The other two holes 3b, 3c respectively communicate with the interiors of the two sheaths 2 and constitute distal end portions of the channels 1a.

The distal end (first end) 2a of each of the sheaths 2 has a projection port 2c from which the treatment tool 20 projects, and the proximal end (second end) 2b of each of the sheaths 2 has an insertion port 2d into which the treatment tool 20 is inserted. The sheaths 2 are each divided into two regions 61, 62 in the longitudinal direction.

The first region 61 including the distal end 2a is a portion inserted into a body cavity when the guide tube 1 is used and has flexibility so as to be bendable along the shape of the body cavity. In a state in which the insertion portion 21 is disposed in the first region 61, the first region 61 can bend in accordance with a force received from the bending portion 14.

The second region 62 including the proximal end 2b is a portion disposed outside of the body when the guide tube 1 is used. The second region 62 is a high-rigidity portion having higher rigidity than the rigidity of the first region 61 and can maintain the shape thereof against a force received from the insertion portion 21 disposed in the second region 62. In other words, in the second region 62, bending and twisting of the insertion portion 21 are suppressed by the rigidity of the second region 62. By doing so, the advancing/retracting and rotating movements applied to the proximal end of the insertion portion 21 from the manipulating portion 23 are efficiently transmitted in the second region 62 toward the distal end of the insertion portion 21. The rigidity of the second region 62 is preferably higher than the rigidity of the insertion portion 21.

The length of the first region 61 is greater than the length from the distal end of the insertion portion 11 to the proximal end of the bending portion 14, and the first region 61 is parallel to the bending portion 14 over the entire length of the bending portion 14.

In addition, the lengths of the first region 61 and the second region 62 are determined on the basis of the total length of the sheath 2 and the length of the sheath 2 inserted into a body cavity (the length of the body cavity from an entrance of the body cavity to an affected site). In the case of the guide tube 1 for the large intestine, the total length of the sheath 2 from the distal end 2a to the proximal end 2b is 1200-1900 mm. In this case, in consideration of the general length of the sheath 2 inserted into the large intestine, the length of the second region 62 is preferably 20-30% of the total length of the sheath 2.

The fixing portion 4 is provided at a boundary 2e between the first region 61 and the second region 62 or in the vicinity of the boundary 2e. In other words, the first region 61 is a region closer to the distal end than the fixing portion 4 is, and the second region 62 is a region closer to the proximal end than the fixing portion 4 is. As described above, the length of the first region 61 is greater than the length from the distal end of the endoscope 10 to the proximal end of the bending portion 14, and thus, the fixing portion 4 is fixed to the insertion portion 11 at a position closer to the proximal end than the bending portion 14 is.

FIG. 2B shows a configuration example of the fixing portion 4. The fixing portion 4 has a recessed portion 4a into which the insertion portion 11 is press-fitted in a radial direction and two holes 4b, 4c into which the sheaths 2 are respectively inserted in the longitudinal direction. The recessed portion 4a and the holes 4b, 4c are parallel to each other and extend from a distal end surface to a proximal end surface of the fixing portion 4. The inner surfaces of the holes 4b, 4c are fixed to the outer surfaces of the sheaths 2 by means of an adhesive. As a result of the insertion portion 11 being press-fitted into the recessed portion 4a, the fixing portion 4 is fixed to the insertion portion 11 in a snap-fit manner. A finger slip stopper 4d may be provided on the outer surface of the fixing portion 4 so that a user can easily hold the fixing portion 4 with his/her fingers.

As shown in FIG. 2A, the sheaths 2 are each fixed to the insertion portion 11 at two locations, namely, the distal end 2a and the boundary 2e, by means of the cap 3 and the fixing portion 4. Therefore, the movements of the first regions 61 of the respective sheaths 2 with respect to the insertion portion 11 are restricted, and the second regions 62 of the respective sheaths 2 can be freely moved with respect to the insertion portion 11.

The distal end of the holder 5 is directly connected to the proximal end 2b of the sheath 2, and the second region 62 is continuous with the holder 5. The inner diameter of the holder 5 is larger than the inner diameter of the second region 62, and an annular abutment surface against which the distal end of the shaft 23a abuts is formed at the distal end of the holder 5.

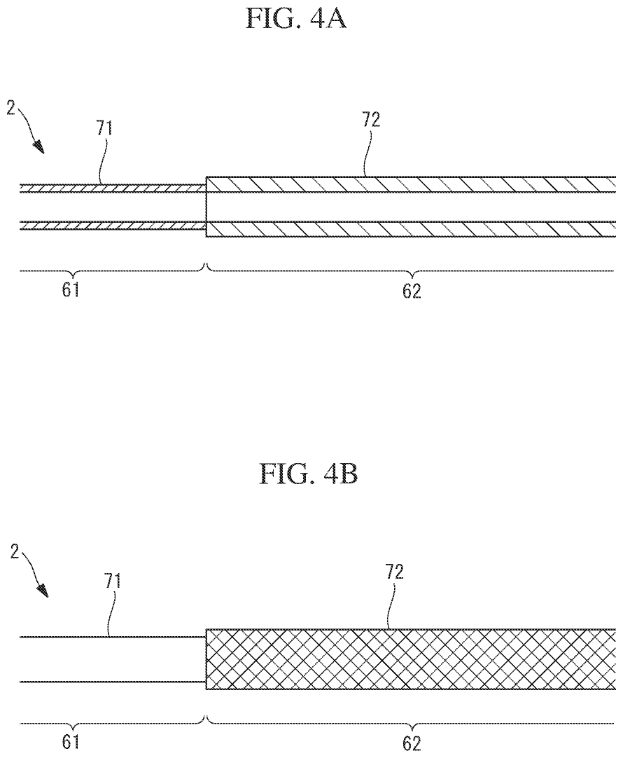

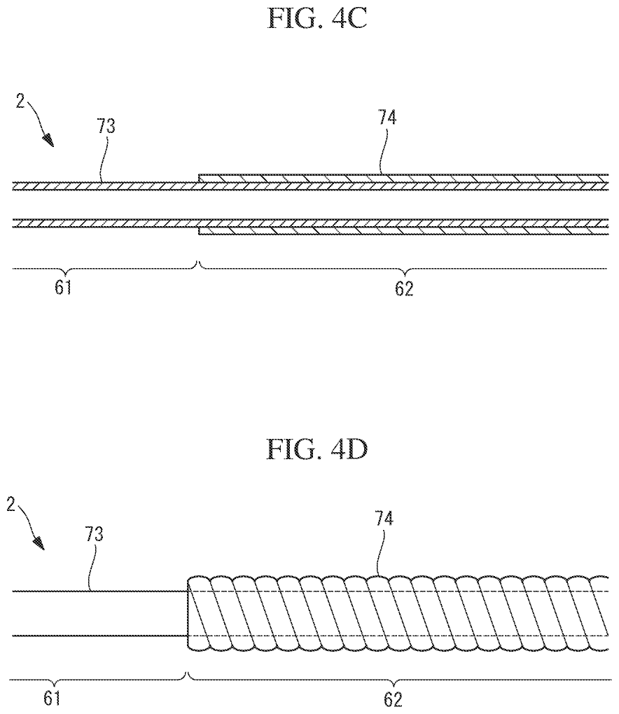

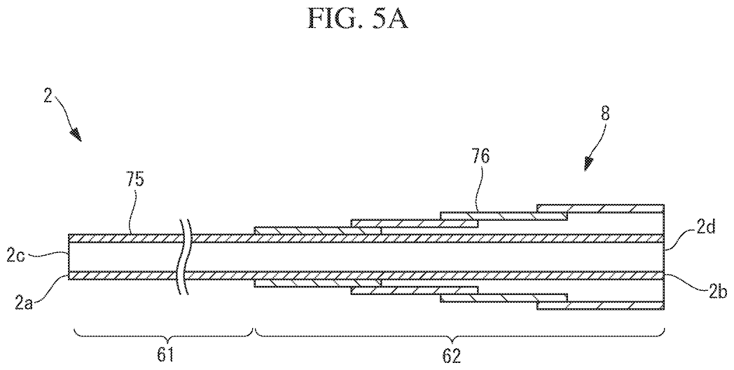

FIGS. 4A to 4D show configuration examples of the first region 61 and the second region 62 of the sheath 2.

The sheaths 2 in FIGS. 4A and 4B are each composed of two tubes 71, 72 that are connected to each other in the longitudinal direction. The first region 61 is composed of the flexible tube 71. The second region 62 is composed of the tube 72 having higher rigidity than the rigidity of the tube 71. The tube 72 in FIG. 4A has a greater wall thickness than the wall thickness of the tube 71. The tube 72 in FIG. 4B is a braided tube in which a netted structure composed of fibers is formed in the wall thereof.

The sheath 2 in FIG. 4C is composed of two tubes 73, 74 that are coaxially arranged. The inner tube (first tube) 73 has flexibility. The outer tube (second tube) 74 covers the inner tube 73 from the proximal end to an intermediate position in the longitudinal direction. The first region 61 is composed of the one inner tube 73. The second region 62 is composed of the two tubes 73, 74.

The outer tube 74 preferably has higher rigidity than the rigidity of the inner tube 73. For example, the tube 74 is a heat-shrinkable tube. Alternatively, the tube 74 may be a rigid tube, for example, a metal tube. As shown in FIG. 4D, the tube 74 may be a flexible tube capable of bending into an arbitrary shape and also capable of retaining the bent shape.

Next, the operation of the thus-configured guide tube 1 and treatment system 100 will be described by illustrating the case in which an affected site in the large intestine is treated.

As shown in FIG. 1, the guide tube 1 is attached to the endoscope 10 prior to insertion of the guide tube 1 into the large intestine. In a guide-tube attachment method, the cap 3 is first attached to the distal end portion of the insertion portion 11 of the endoscope 10. Next, the fixing portion 4 is fixed to the insertion portion 11, thereby fixing the positions between the first regions 61 and the second regions 62 of the respective sheaths 2 to the insertion portion 11 at a position between the bending portion 14 and the operating portion 12. By doing so, the two sheaths 2 are arranged so as to be parallel to the insertion portion 11.

The scopist A inserts the insertion portion 11, together with the two sheaths 2, into the large intestine of the patient P from the anus to the affected site. Because the lengths of the first regions 61 are designed to be the lengths of the sheaths 2 inserted to the affected site, the fixing portion 4 is disposed in the vicinity of the anus in a state in which the sheaths 2 are inserted to the affected site. In other words, the first regions 61 having low rigidity are disposed in the large intestine and the second regions 62 having high rigidity are disposed outside of the body.

Next, the operator B inserts the treatment tools 20 through the channels 1a from the proximal ends of the holders 5 to the affected site. Next, the operator B treats the affected site with the end effectors 22 while observing the affected site and the end effectors 22 with the endoscope 10.

Here, the operator B can change the orientation of the distal end of the insertion portion 11 with respect to the affected site by operating the operating portion 12 to bend the bending portion 14 in the large intestine.

In addition, the operator B can change the positions and orientations of the end effectors 22 in the large intestine by operating the manipulating portions 23. Specifically, the operator B can advance the end effectors 22 by advancing the manipulating portions 23 and can retract the end effectors 22 by retracting the manipulating portions 23. In addition, the operator B can rotate the end effectors 22 by rotating the manipulating portions 23 about the longitudinal axes.

As described above, with this embodiment, the first region 61 disposed in a body cavity has high flexibility. Therefore, the sheath 2 can be easily inserted into the body cavity along the curved shape of the body cavity. In addition, the first region 61 having high flexibility is disposed parallel to the bending portion 14 in the body cavity. Therefore, it is possible to bend the bending portion 14 together with the first region 61 and to realize good operability of the bending portion 14.

In addition, the second region 62 disposed outside of the body has higher rigidity than the first region 61. Therefore, the second region 62 is not bent or twisted due to the force received from the insertion portion 21 of the treatment tool 20, and bending and twisting of the insertion portion 21 in the second region 62 are suppressed by the rigidity of the second region 62. In other words, the advancing/retracting and rotating movements applied to the proximal end of the insertion portion 21 from the manipulating portion 23 are transmitted to the end effector 22 with high efficiency via the insertion portion 21. By doing so, it is possible to realize good operability of the treatment tool 20.

In this embodiment, the guide tube 1 may further include a length adjusting mechanism 8 for adjusting the length ratio between the first region 61 and the second region 62.

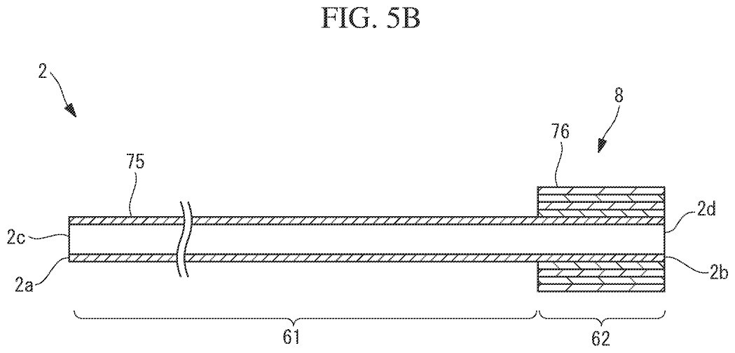

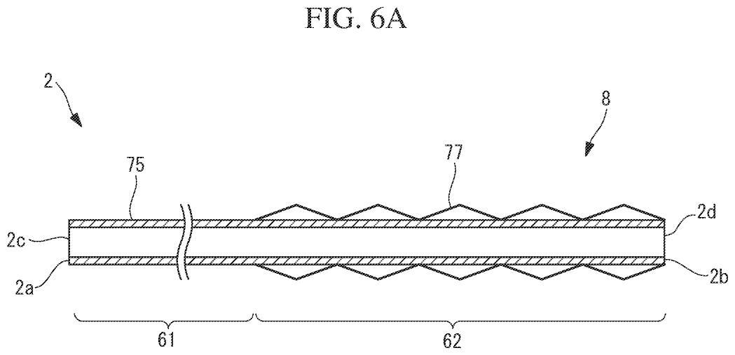

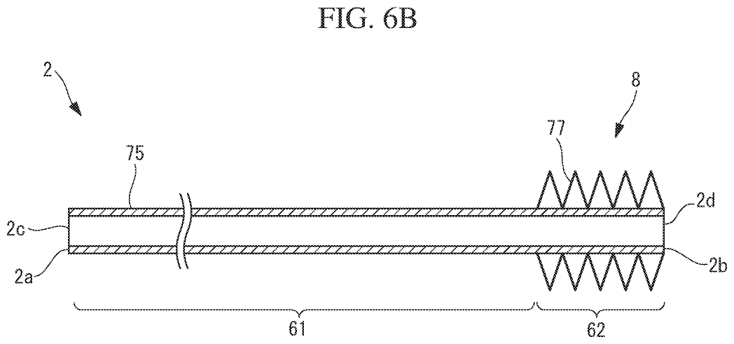

FIGS. 5A and 5B show an example of the length adjusting mechanism 8. In the example, the length adjusting mechanism 8 is composed of a tube 76 having a telescope structure capable of extending and contracting in the longitudinal direction. FIGS. 6A and 6B show another example of the length adjusting mechanism 8. In the other example, the length adjusting mechanism 8 is composed of a tube 77 having a bellows structure capable of extending and contracting in the longitudinal direction.

The tube 76, 77 is arranged coaxially with a flexible tube 75 and covers the tube 75 from the proximal end to an intermediate position in the longitudinal direction. The first region 61 is composed of the one tube 75 and the second region 62 is composed of the two tubes 75, 76 or 75, 77. The proximal end of the tube 76, 77 is fixed to the proximal end of the tube 75. As a result of extension and contraction of the tube 76, 77, the distal end of the tube 76, 77 moves in the longitudinal direction with respect to the tube 75, and thus, the length ratio between the first region 61 and the second region 62 changes.

The length of the sheath 2 inserted into a body cavity, that is, the optimum lengths of the first region 61 and the second region 62, differs according to the position of an affected site in the body cavity. With the guide tube 1 including the length adjusting mechanism 8, it is possible to easily adjust the length of the first region 61 to a length suitable for the length inserted into the body cavity. The adjustment of the length ratio between the first region 61 and the second region 62 is performed before or after the sheath 2 is inserted into the body cavity.

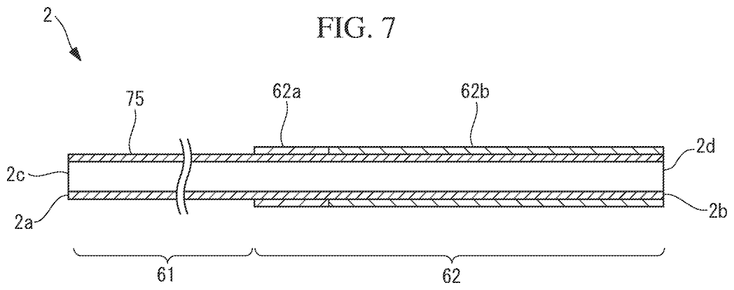

Although the rigidity of the second region 62 may be uniform over the entire length thereof in this embodiment, as shown in FIG. 7, the rigidity of the second region 62 may change in the longitudinal direction.

There is a possibility that a distal end portion of the second region 62 is inserted into a body cavity depending on the situation. Therefore, for example, as shown in FIG. 7, a distal end portion 62a of the second region 62 may have lower rigidity than another portion 62b of the second region 62. In addition, the rigidity of the distal end portion 62a may decrease from the proximal end toward the distal end in a continuous or stepwise manner.

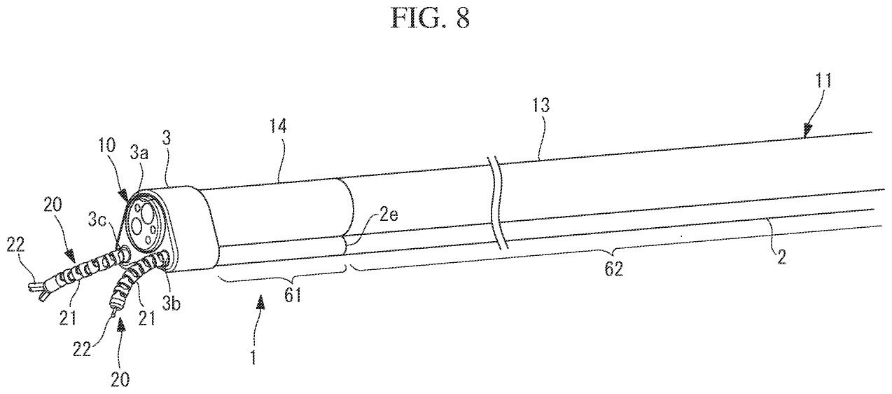

In this embodiment, the boundary 2e between the first region 61 and the second region 62 is provided at such a position that the boundary 2e is disposed at an entrance (for example, the anus) of a body cavity of a patient P when the guide tube 1 is used, or is provided in the vicinity of said position. Alternatively, as shown in FIG. 8, the boundary 2e may be provided at the proximal end of the bending portion 14 or in the vicinity thereof.

With the guide tube 1 in FIG. 8, the first region 61 having high flexibility is disposed parallel to the bending portion 14, and thus, it is possible to realize good operability of the bending portion 14. In addition, because a portion other than the first region 61 of the sheath 2 is the second region 62 having high rigidity, it is possible to realize good operability of the treatment tool 20.

As shown in FIG. 8, the second region 62 may have such a length that the second region 62 is parallel to the flexible portion 13 of the endoscope 10 over substantially the entire length thereof, and the second region 62 may also be inserted into the body cavity.

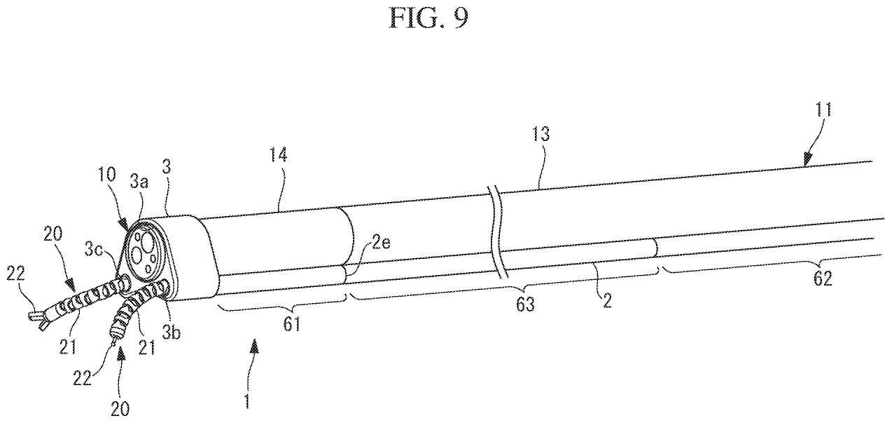

Alternatively, as shown in FIG. 9, the second region 62 may be composed only of a portion disposed outside of the body when the guide tube 1 is used. In this case, a third region 63 having flexibility is further provided between the first region 61 and the second region 62. In other words, the sheath 2 is divided into the three regions 61, 62, 63 in the longitudinal direction. The third region 63 has rigidity higher than the rigidity of the first region 61 and lower than the rigidity of the second region 62. The portion of the sheath 2 inserted into the body cavity is composed of the regions 61, 63 having flexibility, and thus, it is possible to enhance the insertability of the sheath 2 into the body cavity.

Although the guide tube 1 includes the cap 3 and the fixing portion 4 in this embodiment, alternatively, at least one of the cap 3 and the fixing portion 4 need not be provided.

For example, the insertion portion 11 of the endoscope 10 and the sheath 2 may be inserted into a body cavity in a state in which only the distal ends are fixed to each other by means of the cap 3. Alternatively, the insertion portion 11 of the endoscope 10 and the sheath 2 may be separately inserted into a body cavity, and the sheath 2 may be movable with respect to the insertion portion 11 in the body cavity.

The above-described embodiment also leads to the following aspects.

An aspect of the present invention is a guide tube including an elongated sheath into which an elongated treatment tool is inserted so as to be movable in a longitudinal direction and that is arranged so as to be parallel to an endoscope, wherein: the sheath has a first end having a projection port from which the treatment tool projects and a second end having an insertion port into which the treatment tool can be inserted and is divided, in the longitudinal direction, into a first region including the first end and having flexibility and a second region including the second end; and the second region has a high-rigidity portion having higher rigidity than a rigidity of the first region.

With this aspect, the sheath is inserted into a body cavity along with the endoscope from an entrance of the body cavity, and the distal end of the endoscope and the first end of the sheath are disposed in the vicinity of an affected site in the body cavity. Next, the treatment tool is inserted into the sheath from the insertion port at the second end disposed outside of the body, and a distal end portion of the treatment tool projects from the projection port at the first end. Next, treatment of the affected site is performed by the treatment tool while the affected site and the treatment tool are observed with the endoscope.

In this case, the first region that is a distal-end side portion of the sheath and that has flexibility is disposed parallel to a bending portion of the endoscope in the body cavity. Therefore, it is possible to bend the sheath together with the bending portion of the endoscope and to realize good operability of the bending portion of the endoscope.

In addition, the second region of the sheath, which is disposed closer to the proximal end than the first region is, is less likely to be deformed due to a force received from the treatment tool, and thus, an advancing/retracting or rotating movement applied to the proximal end of the treatment tool outside of the body is efficiently transmitted in the second region toward the distal end of the treatment tool. Therefore, it is possible to realize good operability of the treatment tool.

In the abovementioned aspect, a fixing portion that is attached to the sheath at a position between the first region and the second region may be provided, and the fixing portion may be fixed to the endoscope at a position closer to a proximal end than a bending portion of the endoscope is.

With this configuration, the sheath can be fixed to the endoscope by the fixing portion at such a position that bending of the bending portion and the first region is not hindered.

In the abovementioned aspect, a cylindrical holder that is connected to the second end and that has a larger inner diameter than an inner diameter of the sheath may be provided.

With this configuration, a manipulating portion of the treatment tool can be supported by the holder.

In the abovementioned aspect, the high-rigidity portion may be continuous with the holder.

With this configuration, the movement of the treatment tool in the holder is transmitted more efficiently to the distal-end side of the treatment tool. Therefore, it is possible to further enhance the operability of the treatment tool.

In the abovementioned aspect, a total length of the sheath from the first end to the second end may be 1200-1900 mm, and a length of the high-rigidity portion may be 20-30% of the total length of the sheath.

With this configuration, the respective lengths of the first region and the second region can be set to lengths suitable for practical use.

In the abovementioned aspect, the high-rigidity portion may include a first tube and a second tube that covers the first tube.

With this configuration, the high-rigidity portion is formed of a two-layer structure tube. Thus, it is possible to easily manufacture the high-rigidity portion.

In the abovementioned aspect, a rigidity of the second tube may be higher than a rigidity of the first tube.

With this configuration, it is possible to further increase the rigidity of the high-rigidity portion.

Another aspect of the present invention is a treatment system including: a guide tube described above; and an endoscope, wherein the second region of the sheath is movable with respect to the endoscope.

Another aspect of the present invention is a treatment system including: a guide tube described above; and a treatment tool having an insertion portion that can be inserted into the sheath of the guide tube, wherein a rigidity of the high-rigidity portion is higher than a rigidity of the insertion portion.

Another aspect of the present invention is a guide-tube attachment method for attaching a guide tube to an endoscope, wherein: the guide tube includes an elongated sheath into which an elongated treatment tool is inserted so as to be movable in a longitudinal direction and that is arranged so as to be parallel to the endoscope, the sheath has a first end having a projection port from which the treatment tool projects and a second end having an insertion port into which the treatment tool can be inserted and is divided, in the longitudinal direction, into a first region including the first end and having flexibility and a second region including the second end, and the second region has a high-rigidity portion having higher rigidity than a rigidity of the first region; and the method includes fixing a position between the first region and the second region of the sheath to the endoscope, at a position closer to a proximal end than a bending portion of the endoscope is and closer to a distal end than an operating portion of the endoscope is.

### REFERENCE SIGNS LIST

1 guide tube
1a channel
sheath
2a distal end (first end)
2b proximal end (second end)
3 cap
4 fixing portion
5 holder
61 first region
62 second region, high-rigidity portion
71, 72, 73, 74, 75, 76 tube
8 length adjusting mechanism
10 endoscope
11 insertion portion
12 operating portion
13 flexible portion
14 bending portion
20 treatment tool
21 insertion portion
22 end effector
23 manipulating portion
30 bed
40 arm
50 display
100 treatment system

1. A guide tube comprising an elongated sheath into which an elongated treatment tool is inserted so as to be movable in a longitudinal direction and that is arranged so as to be parallel to an endoscope, wherein:
the sheath has a first end having a projection port from which the treatment tool projects and a second end having an insertion port into which the treatment tool is inserted and is divided, in the longitudinal direction, into a first region including the first end and having flexibility and a second region including the second end;
the second region has a high-rigidity portion having higher rigidity than a rigidity of the first region; and
a lengths of the first region is not greater than a length of the second region.

2. The guide tube according to claim 1, further comprising a fixing portion that is attached to the sheath at a position between the first region and the second region,
wherein the fixing portion is configured to be fixed to the endoscope at a position closer to a proximal end than a bending portion of the endoscope is.

3. The guide tube according to claim 1, further comprising a fixing portion that is attached to the sheath at a position overlapping a boundary between the first region and the second region,
wherein the fixing portion fixes the sheath to the endoscope.

4. The guide tube according to claim 1, further comprising a cylindrical holder that is connected to the second end and that has a larger inner diameter than an inner diameter of the sheath.

5. The guide tube according to claim 4, wherein the high-rigidity portion is continuous with the holder.

6. The guide tube according to claim 1, wherein a total length of the sheath from the first end to the second end is 1200-1900 mm, and
a length of the high-rigidity portion is 20-30% of the total length of the sheath.

7. The guide tube according to claim 1, wherein the high-rigidity portion includes a first tube and a second tube that covers the first tube.

8. The guide tube according to claim 7, wherein a rigidity of the second tube is higher than a rigidity of the first tube.

9. A treatment system comprising:
a guide tube according to claim 1; and
an endoscope,
wherein the second region of the sheath is movable with respect to the endoscope.

10. A treatment system comprising:
a guide tube according to claim 1; and
a treatment tool having an insertion portion that is inserted into the sheath of the guide tube,
wherein a rigidity of the high-rigidity portion is higher than a rigidity of the insertion portion.

11. A guide tube comprising an elongated sheath into which an elongated treatment tool is inserted in a longitudinal direction and that is arranged so as to be parallel to an endoscope, wherein:
the sheath has a first end from which the treatment tool projects and a second end into which the treatment tool is inserted and is divided, in the longitudinal direction, into a first region including the first end and having flexibility and a second region including the second end;
the second region has a high-rigidity portion having higher rigidity than a rigidity of the first region; and
the high-rigidity portion includes a length adjusting mechanism.

12. The guide tube according to claim 11, wherein the length adjusting mechanism adjusts a length by being folded from a distal end toward a proximal end.

13. A treatment method using a guide tube including an elongated sheath into which an elongated treatment tool is inserted in a longitudinal direction and that is arranged so as to be parallel to an endoscope, the sheath being divided, in the longitudinal direction, into a first region including a first end from which the treatment tool projects and having flexibility, and a second region including a second end into which the treatment tool is inserted and having a high-rigidity portion having higher rigidity than a rigidity of the first region, the method comprising:
inserting only the first region into a body cavity; and
rotationally operating the treatment tool after the inserting.
